(19) United States
(12) Patent Application Publication
(10) Pub. No.: US 2026/0242535 A1
(43) Pub. Date: Aug. 20, 2026

(54) PROCESS FOR SYNTHESIS OF EPOXY RESIN FROM VANILLIN-BASED DIALDEHYDES, AMINOPHENOL DERIVATIVES, AND EPICHLOROHYDRIN

(71) Applicants: KOREA INSTITUTE OF SCIENCE AND TECHNOLOGY, Seoul (KR); KUMAUN UNIVERSITY, Nainital (IN)

(72) Inventors: Yongchae JUNG, Seoul (KR); Chetna TEWARI, Seoul (KR); Youngnam KIM, Seoul (KR); Kundan Singh RAWAT, Nainital (IN); Nanda Gopal SAHOO, Nainital (IN)

(73) Assignees: KOREA INSTITUTE OF SCIENCE AND TECHNOLOGY, Seoul (KR); KUMAUN UNIVERSITY, Nainital (IN)

(21) Appl. No.: 19/385,510

(22) Filed: Nov. 11, 2025

(30) Foreign Application Priority Data
Feb. 17, 2025 (IN) 202511013621
Sep. 9, 2025 (KR) 10-2025-0128171

(51) Int. Cl. C08G 59/06; C07C 251/24; C07D 303/24

(52) U.S. Cl. CPC C08G 59/063; C07C 251/24; C07D 303/24; C08G 2261/3424

(57) ABSTRACT

The present disclosure relates to a chemical process and in particularly, relates to a process for synthesis of epoxy resin from vanillin-based dialdehydes, aminophenol derivatives, and epichlorohydrin. The process comprising preparing a dialdehyde compound (DAC) by dissolving a vanillin derivative in an anhydrous dimethylformamide (DMF); preparing a diphenol compound (DPC) by dissolving the dialdehyde compound (DAC) in an absolute ethanol; mixing the diphenol compound (DPC) with an epichlorohydrin (ECH) in a presence of a tetrabutylammonium bromide (TBAB) to form a reaction mixture; adding an aqueous sodium hydroxide (NaOH) solution dropwise to induce ring closure and promote epoxy formation; extracting the epoxy resin by using a dichloromethane (DCM) or an ethyl acetate and drying the epoxy resin over an anhydrous sodium sulfate (Na₂SO₄) to remove residual moisture; evaporating solvent under reduced pressure in a rotavapour, yielding a high-purity dual imine functionalized epoxy resin suitable for advanced material applications.

### CROSS-REFERENCE TO RELATED APPLICATION

This application claims the benefit under 35 USC 119 (a) of Indian Patent Application number 202511013621, filed with the Indian Intellectual Property Office on Feb. 17, 2025 and Korean Patent Application No. 10-2025-0128171, filed with the Korean Intellectual Property Office on Sep. 9, 2025, the disclosure of which is incorporated herein by reference in its entirety.

### FIELD OF THE INVENTION

The present disclosure relates to a chemical process and in particularly, relates to a process for synthesis of epoxy resin from vanillin-based dialdehydes, aminophenol derivatives, and epichlorohydrin. The present invention relates to reprocessable and self-healing epoxy resins incorporating dual imine functionalization. These epoxy resins, derived from bio-based vanillin precursors, exhibit excellent recyclability, high mechanical strength, and superior thermal stability. The invention finds utility in coatings, adhesives, aerospace, automotive, and composite material applications.

### BACKGROUND OF THE INVENTION

The irreversible crosslinking of conventional epoxy resins makes them difficult to recycle, resulting in significant environmental concerns. To address this challenge, researchers have explored dynamic covalent networks, particularly those based on imine chemistry, due to their reversibility under controlled conditions. However, previously reported epoxy vitrimer systems often suffer from limited reprocessing efficiency or require catalysts for bond exchange reactions.

Vanillin, a widely available lignin derivative, offers a renewable alternative for synthesizing epoxy resins with improved sustainability. The introduction of dual imine linkages in epoxy networks enhances their recyclability and self-healing properties without compromising structural integrity. Unlike the prior art, such as CN 117164816 A, which describes Schiff-base epoxy resins with limited recyclability, the present invention achieves multiple reprocessing cycles while maintaining mechanical performance.

In the view of the forgoing discussion, it is clearly portrayed that there is a need to have a process for synthesis of epoxy resin from vanillin-based dialdehydes, aminophenol derivatives, and epichlorohydrin.

### SUMMARY OF THE INVENTION

The present disclosure relates to a chemical process and in particularly, relates to a process for synthesis of epoxy resin from vanillin-based dialdehydes, aminophenol derivatives, and epichlorohydrin. The present invention relates to reprocessable and self-healing epoxy resins incorporating dual imine functionalization. These epoxy resins, derived from bio-based vanillin precursors, exhibit excellent recyclability, high mechanical strength, and superior thermal stability. The invention finds utility in coatings, adhesives, aerospace, automotive, and composite material applications.

In an embodiment, a process for synthesis of epoxy resin from vanillin-based dialdehydes, aminophenol derivatives and epichlorohydrin is provided. The process comprising: preparing a dialdehyde compound (DAC) by dissolving a vanillin derivative in an anhydrous dimethylformamide (DMF); preparing a diphenol compound (DPC) by dissolving the dialdehyde compound (DAC) in an absolute ethanol; mixing the diphenol compound (DPC) with an epichlorohydrin (ECH) in a presence of a tetrabutylammonium bromide (TBAB) to form a reaction mixture; adding an aqueous sodium hydroxide (NaOH) solution dropwise to induce ring closure and promote epoxy formation; extracting the epoxy resin by using a dichloromethane (DCM) or an ethyl acetate and drying the epoxy resin over an anhydrous sodium sulfate (Na₂SO₄) to remove residual moisture; evaporating solvent under reduced pressure in a rotavapour, yielding a high-purity dual imine functionalized epoxy resin suitable for advanced material applications.

In another embodiment, the preparing the dialdehyde compound (DAC) by using the vanillin derivative in the anhydrous dimethylformamide (DMF), said preparing process comprising: dissolving the vanillin derivative in the anhydrous dimethylformamide (DMF) to form a mixture and stirring the mixture to ensure complete solubility; adding an alkyl dibromides and potassium carbonate (K₂CO₃) to the mixture; heating the mixture at 70° C. for 24 hours under an inert nitrogen atmosphere to prevent oxidation and unwanted side reactions.

In another embodiment, the preparing the dialdehyde compound (DAC) by using the vanillin derivative in the anhydrous dimethylformamide (DMF), said preparing process further comprising: cooling the mixture to room temperature and pouring the mixture into a deionized water to precipitate the dialdehyde compound; vacuum filtering the precipitate of the dialdehyde compound and washing thoroughly to remove residual salts and unreacted reagents; purifying the precipitate of the dialdehyde compound via recrystallization in an ethanol or methanol to obtain a high-purity dialdehyde compound.

In another embodiment, the potassium carbonate (K₂CO₃) acts as a base in the mixture to facilitate a nucleophilic substitution reaction.

In another embodiment, the preparing the diphenol compound (DPC) by dissolving the dialdehyde compound (DAC) in the absolute ethanol, said preparing process comprising: dissolving the dialdehyde compound (DAC) in the absolute ethanol to form a mixture and stirring the mixture to ensure complete dissolution; adding a p-Aminophenol gradually under continuous stirring into the mixture; heating the mixture to 80° C. under reflux for 24 hours to form an imine (—C═N—) linkages while ensuring complete conversion of starting materials.

In another embodiment, the preparing the diphenol compound (DPC) by dissolving the dialdehyde compound (DAC) in the absolute ethanol, said preparing process further comprising: cooling the mixture to room temperature and subjecting the mixture to vacuum filtration to separate a solid diphenol compound; washing the solid diphenol compound thoroughly with a cold ethanol to remove any unreacted residues and by-products; and vacuum drying the diphenol intermediate at 50-60° C. to obtain a high-purity diphenol compound (DPC).

In another embodiment, a condensation reaction is facilitated between aldehyde and amine functional groups.

In another embodiment, the tetrabutylammonium bromide (TBAB) acts as a phase-transfer catalyst to enhance an etherification reaction.

In another embodiment, the reaction mixture is maintained at 100-110° C. under continuous stirring to ensure complete functionalization.

In another embodiment, temperature of the reaction mixture is gradually increased to 50-60° C. to facilitate polymerization.

In addition, in an embodiment, the epoxy resin may have the following chemical formula:

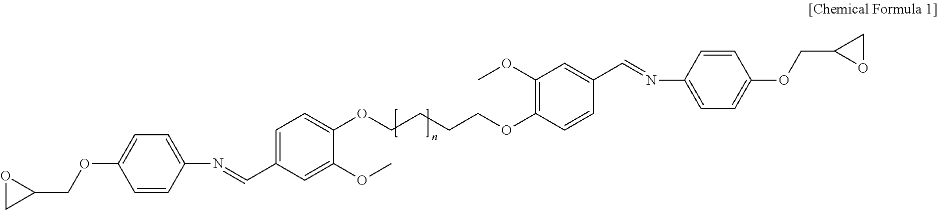

In another embodiment, the epoxy resin may correspond to one among 1,2-bis{2-[(4-(2,3-epoxypropoxy)benzylidene)amino]-5-methoxyphenoxy}ethane, 1,3-bis{2-[(4-(2,3-epoxypropoxy)benzylidene)amino]-5-methoxyphenoxy}propane, and 1,4-bis{2-[(4-(2,3-epoxypropoxy)benzylidene)amino]-5-methoxyphenoxy}butane.

An object of the present disclosure is to fabricate epoxy resin comprises a dual imine functionalized structure derived from vanillin-based precursors.

Another object of the present disclosure is to provide epoxy resin material, which is highly resistant to thermal degradation, with onset temperatures exceeding 250° C.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates synthesis of diimine functionalized epoxy (DIFE) in accordance with an embodiment of the present invention;

FIG. 2 illustrates 1H NMR of 4,4′-(butane-1,4-diylbis(oxy))bis(3-methoxybenzaldehyde) in CDCl3 in accordance with an embodiment of the present invention;

FIG. 3 illustrates 1H NMR of 4,4′-(((1Z,1′Z)-((butane-1,4-diylbis(oxy))bis(3-methoxy-4,1-phenylene))bis(methaneylylidene))bis(azaneylylidene))diphenol in DMSO-d6 in accordance with an embodiment of the present invention;

FIG. 4 illustrates 1H NMR of (1E,1′E)-1,1′-((butane-1,4-diylbis(oxy))bis(3-methoxy-4,1-phenylene))bis(N-(4-(oxiran-2-ylmethoxy)phenyl) methanimine) in CDCl3 in accordance with an embodiment of the present invention;

FIG. 5 illustrates DSC, TGA and DMA data of epoxy Films in accordance with an embodiment of the present invention;

FIG. 6 illustrates Stress-strain curves of different epoxy with jeffamine D230 in accordance with an embodiment of the present invention;

FIG. 7 illustrates 1,4 epoxy D230 Film Reprocessing up to 10 cycles in accordance with an embodiment of the present invention;

FIG. 8 illustrates self-healing of 1,4 epoxy D230 Film in accordance with an embodiment of the present invention;

FIG. 9 illustrates a table which describe differences of the present invention with Prior Art (CN 117164816 A& Others) in accordance with an embodiment of the present invention; and

FIG. 10 illustrates a flow chart for process for synthesis of epoxy resin from vanillin-based dialdehydes, aminophenol derivatives, and epichlorohydrinin accordance with an embodiment of the present disclosure.

Further, skilled artisans will appreciate that elements in the drawings are illustrated for simplicity and may not have necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

The present disclosure fabricates epoxy resin comprises a dual imine functionalized structure derived from vanillin-based precursors. The resin formulation allows for tunable mechanical properties by varying the carbon linker chain length (C-2 to C-12). The material exhibits high reprocessability, retaining nearly 100% of its tensile strength after 10 cycles. The glass transition temperature is adjustable between 50° C. and 75° C. based on precursor selection. The resin formulation does not require external catalysts for dynamic covalent bond exchange. The material is highly resistant to thermal degradation, with onset temperatures exceeding 250° C. The storage modulus confirms high-temperature mechanical stability across various formulations. The resin exhibits tensile strengths ranging from 97 MPa to 108 MPa with elongation between 11% and 25%. The resin is synthesized through a sustainable method utilizing bio-based vanillin derivatives. The resin is compatible with various crosslinking agents and hardeners for customizable curing conditions. The epoxy system can be efficiently processed using conventional industrial techniques such as injection molding and 3D printing. The resin is ideal for high-performance applications in coatings, adhesives, and composite materials. The self-healing efficiency of the material is validated through microcrack healing under controlled conditions. The recyclability of the material ensures reduced environmental impact and waste minimization. The formulation enables the development of lightweight, impact-resistant composite structures. The resin is suitable for use in aerospace, automotive, and electronics applications due to its high mechanical durability. The polymer network allows reversible reshaping, making it an excellent candidate for additive manufacturing. The epoxy vitrimer maintains chemical resistance against solvents and environmental stressors. The epoxy resin provides superior adhesion and mechanical interlocking, enhancing its performance in structural applications.

The present invention provides a reprocessable and self-healing epoxy resin comprising dual imine bonds derived from vanillin-based dialdehydes. The polymer film prepared from the resin demonstrates tensile strength retention above 100 MPa after multiple reprocessing cycles. The polymer film exhibits autonomous repair of microcracks at 120° C. under mild pressure conditions. The epoxy resin system eliminates the need for catalysts in bond exchange reactions. The composite material incorporates the epoxy resin for high-impact resistance. A coating formulation utilizes the epoxy resin to provide enhanced durability and reprocessability. A structural adhesive composition comprises the dual imine-functionalized epoxy resin. A high-temperature-resistant epoxy formulation demonstrates thermal stability exceeding 250° C. A method is provided for integrating the epoxy resin into additive manufacturing and 3D printing applications. A method is provided for modifying the crosslinking density of the resin to achieve specific mechanical properties. A recyclable epoxy vitrimer is designed for aerospace and automotive structural applications. A self-healing composite structure uses the epoxy resin to enhance longevity and damage tolerance.

This invention discloses an epoxy resin synthesized from vanillin-based dialdehydes, aminophenol derivatives, and epichlorohydrin. The resins incorporate dynamic imine linkages, allowing: the material to undergo efficient thermal reprocessing up to ten cycles without mechanical degradation; the system to exhibit self-healing functionality, such that material integrity is restored within 30 minutes at 120° C. under mild pressure; and the mechanical properties to be tunable by varying dialdehyde chain lengths, thereby achieving tensile strengths between 97 MPa and 108 MPa with elongation between 11% and 25%.

A detailed synthesis protocol ensures the reproducibility of the resin's unique features, making it a viable alternative to existing thermoset materials.

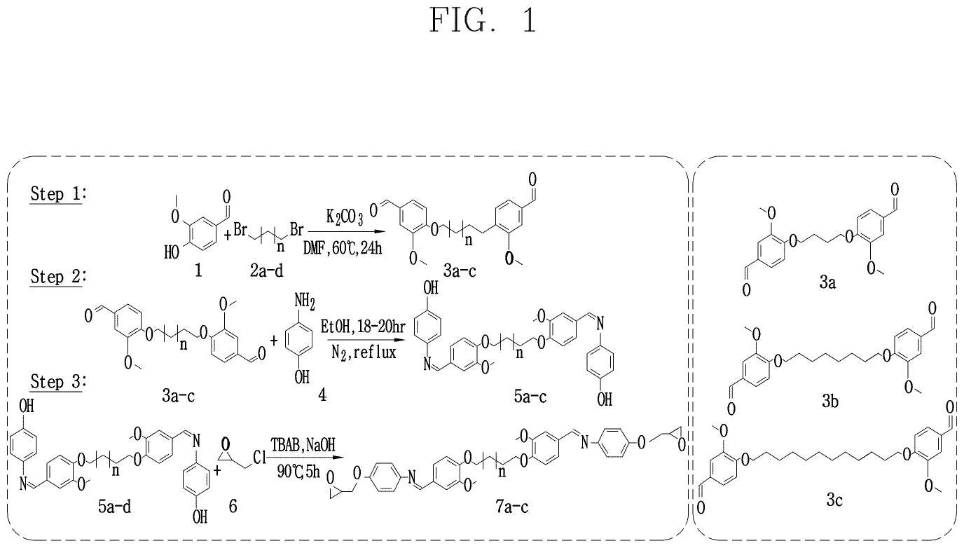

FIG. 1 illustrates synthesis of diimine functionalized epoxy (DIFE) in accordance with an embodiment of the present invention. A detailed synthesis protocol ensures the reproducibility of the resin's unique features, making it a viable alternative to existing thermoset materials.

### Synthesis of Dialdehyde Compound (DA) (3a)

A vanillin derivative (4-hydroxy-3-methoxybenzaldehyde, vanillic aldehyde, vanillin) (1) is dissolved in 1,3-dibromopropane, 1,4-dibromobutane, 1,5-dibromopentane, which correspond to 1,n-dibromoalkanes (2a-d), and stirred to ensure complete solubility. 1,n-dibromoalkane is added gradually to the reaction mixture, followed by the introduction of potassium carbonate (K₂CO₃) as a base to facilitate the nucleophilic substitution reaction. The reaction is conducted at 70° C. for 24 hours under an inert nitrogen atmosphere to prevent oxidation or unwanted side reactions. Once the reaction is complete, the mixture is cooled to room temperature and poured into deionized water to respectively precipitate 1,2-bis[2-formyl-5-methoxyphenoxy]ethane, 1,3-bis[2-formyl-5-methoxyphenoxy]propane, and 1,4-bis[2-formyl-5-methoxyphenoxy]butane, which correspond to dialdehyde compounds (3a-c). The crude product is collected by vacuum filtration, washed thoroughly to remove residual salts and unreacted reagents, and then purified via recrystallization in ethanol or methanol to obtain a high-purity dialdehyde compound with well-defined aldehyde functionalities.

### Synthesis of Diphenol Compound (DPC) (3b)

The dialdehyde compound (DA) (3a-c) is dissolved in absolute ethanol and stirred to ensure complete dissolution. p-Aminophenol (4) is slowly added under continuous stirring, facilitating a condensation reaction between the aldehyde and amine functional groups. The reaction mixture is heated to 80° C. under reflux for 24 hours, allowing the formation of imine (—C═N—) linkages while ensuring complete conversion of the starting materials. Once the reaction is complete, the mixture is cooled to room temperature and subjected to vacuum filtration to respectively separate 1,2-bis{2-[(4-hydroxyphenyl)iminomethyl]-5-methoxyphenoxy}ethane, 1,3-bis{2-[(4-hydroxyphenyl)iminomethyl]-5-methoxyphenoxy}propane, and 1,4-bis{2-[(4-hydroxyphenyl)iminomethyl]-5-methoxyphenoxy}butane, which correspond to solid diphenol compounds (5a-c). The crude product is washed thoroughly with cold ethanol to remove any unreacted residues or by-products. Finally, the diphenol intermediate is vacuum dried at 50-60° C. to obtain a high-purity diphenol compound (DPC), which serves as a key precursor for subsequent epoxy functionalization reaction.

### Epoxy Functionalization (DIFE Synthesis) (3c)

The diphenol compound (5a-c) is reacted with epichlorohydrin (ECH) (6) in the presence of tetrabutylammonium bromide (TBAB), which acts as a phase-transfer catalyst to enhance the etherification reaction. To ensure complete functionalization, 1,2-bis{2-[(4-(2,3-epoxypropoxy)benzylidene)amino]-5-methoxyphenoxy}ethane, 1,3-bis{2-[(4-(2,3-epoxypropoxy)benzylidene)amino]-5-methoxyphenoxy}propane, and 1,4-bis{2-[(4-(2,3-epoxypropoxy)benzylidene)amino]-5-methoxyphenoxy}butane, which correspond to the reaction mixture (7a-c), are continuously stirred at 100-110° C. After the initial reaction, an aqueous sodium hydroxide (NaOH) solution is added dropwise to induce ring closure and promote epoxy formation. The temperature is gradually increased to 50-60° C. to facilitate polymerization. The epoxy resin is then extracted using dichloromethane (DCM) or ethyl acetate, followed by drying over anhydrous sodium sulfate (Na₂SO₄) to remove residual moisture. Finally, the solvent is evaporated under reduced pressure in a rotavapour, yielding a high-purity dual imine functionalized epoxy resin suitable for advanced material applications.

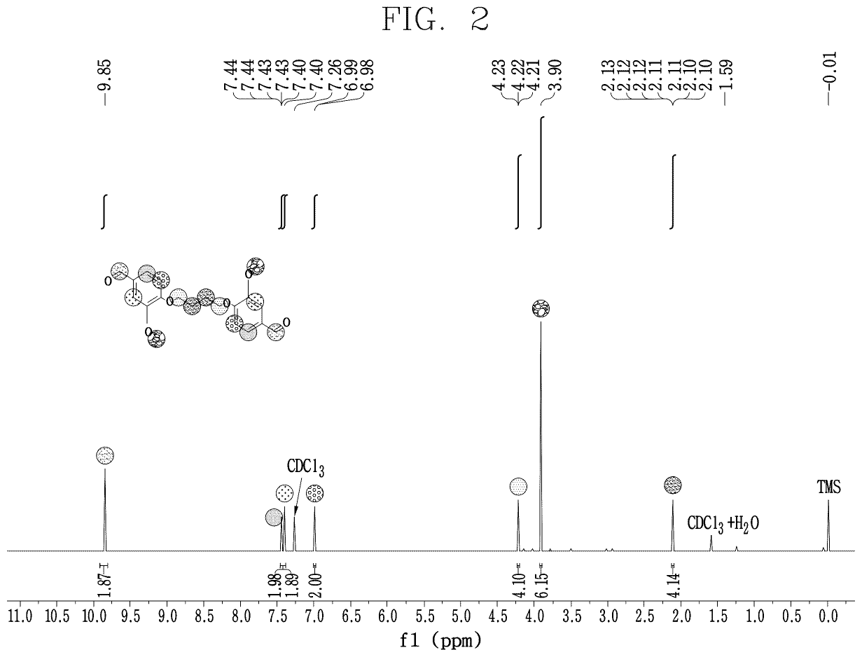

FIG. 2 illustrates 1H NMR of 4,4′-(butane-1,4-diylbis(oxy))bis(3-methoxybenzaldehyde) in CDCl3.

The presented ¹H NMR spectrum (400 MHZ, CDCl₃) successfully confirmed the structure of the synthesized compound. A broad singlet (i.e., single peak) observed at δ 9.85 ppm corresponded to two hydroxyl (—OH) protons, and the integration value was identified as 2H. The aromatic protons appeared as complex multiplets (i.e., multiple peaks) in the range of δ 6.98-7.44 ppm, and the total integration value was 2H. In addition, the methylene protons adjacent to the ether bond (—O—CH₂—) were observed at δ 4.22 ppm as multiplets with an integration value of 4H. The methylene protons at the center of the alkyl chain were detected at δ 2.10 ppm as multiplets with an integration value of 4H. The integral ratio of these key peaks (2:2:4:4) exactly matched the number of protons in the proposed molecular structure.

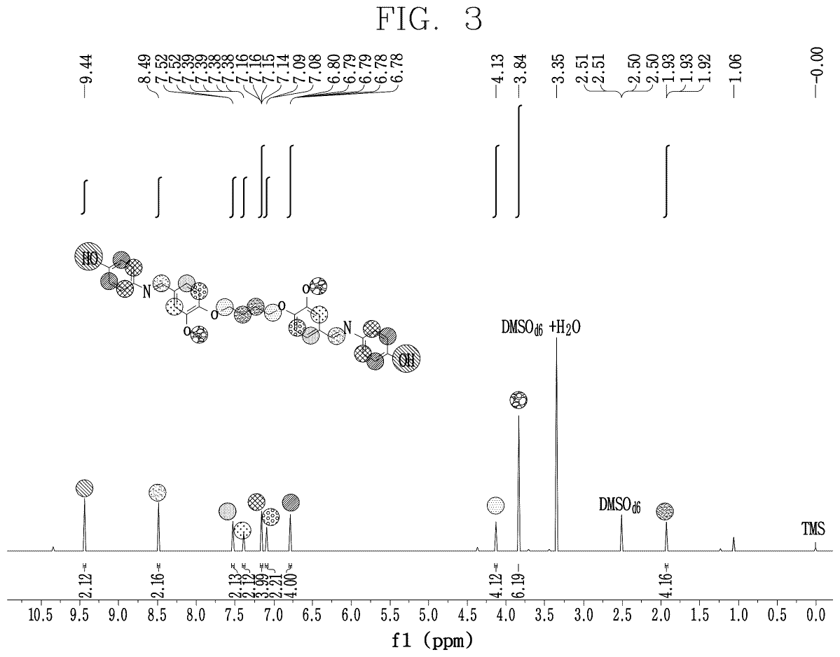

FIG. 3 illustrates 1H NMR of 4,4′-(((1Z,1′Z)-((butane-1,4-diylbis(oxy))bis(3-methoxy-4,1-phenylene))bis(methaneylylidene))bis(azaneylylidene))diphenol in DMSO-d6.

The presented ¹H NMR spectrum (400 MHz, DMSO-d₆) successfully confirmed the structure of the synthesized compound. A broad singlet observed at δ 9.44 ppm corresponded to two hydroxyl (—OH) protons, and the integration value was shown as 2H. In the aromatic region, complex multiplets were observed in the range of δ 6.78-8.49 ppm, and the total integration value corresponded to 8H. In particular, the peak appearing at δ 8.49 ppm was interpreted as being attributable to a pyridine ring proton that was significantly deshielded due to adjacency to a nitrogen atom with an electron-withdrawing effect. The methylene protons adjacent to the ether bond (—O—CH₂—) were observed as multiplets at δ 4.13 ppm, and the integration value was 4H. In addition, the methylene protons adjacent to an amine (—N—CH₂—) were identified as multiplets at δ 3.84 ppm, which also structurally corresponded to 4H. The integration value (6.19) indicated in the spectrum was determined to be an error occurring during the instrumental analysis process, and in reality, it was considered to correspond to 4H. The integral ratio of the main peaks (2H:8H:4H:4H) exactly matched the number of protons in the proposed molecular structure.

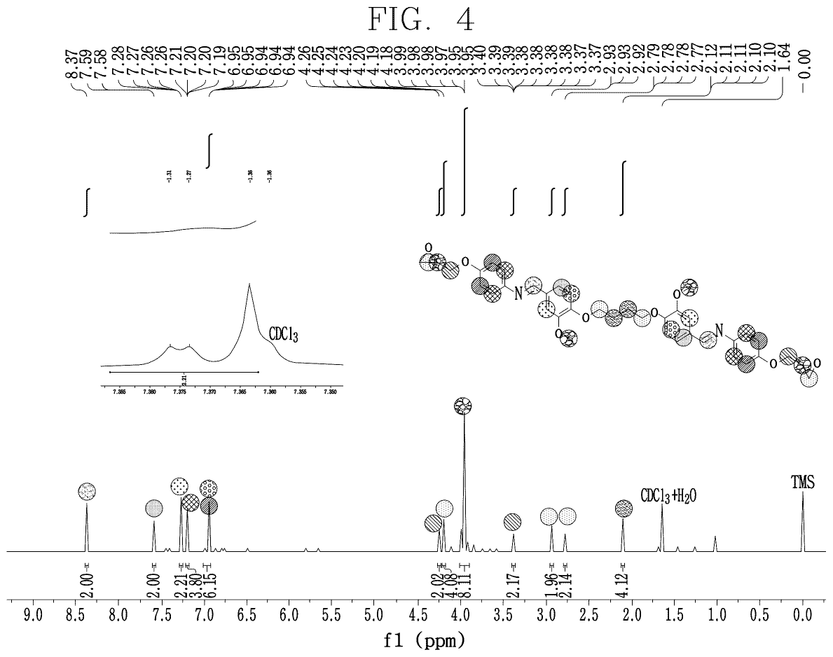

FIG. 4 illustrates ¹H NMR of (1E,1′E)-1,1′-((butane-1,4-diylbis(oxy))bis(3-methoxy-4,1-phenylene))bis(N-(4-(oxiran-2-ylmethoxy)phenyl) methanimine) in CDCl3 in accordance with an embodiment of the present invention.

The presented 1H NMR spectrum (400 MHZ, CDCl₃) was used to confirm the structure of the synthesized compound. The multiplets observed in the range of δ 6.94-8.37 ppm corresponded to aromatic ring protons, and the total integration value was shown as 8H. Among these, the peak observed at δ 8.37 ppm was attributed to 2H protons adjacent to a nitrogen atom, and the remaining complex peaks were assigned to 6H aromatic protons. Two multiplets detected in the range of δ 2.78-2.92 ppm each showed an integration value of 2H, which corresponded to protons of two oxirane rings within the molecule. In the range of δ 3.37-4.26 ppm, signals of central methylene (—CH₂—) protons were observed as complex multiplets. The methylene protons adjacent to an oxygen atom exhibited an integration value of 4H at δ 3.98-4.26 ppm, while the methylene protons adjacent to a nitrogen atom exhibited an integration value of 4H at δ 3.37-3.93 ppm. In addition, signals corresponding to the solvent peak CDCl₃ (δ 7.26 ppm) and residual water (H₂O, δ 1.59 ppm) were also identified. The overall chemical shift values and peak patterns matched the proposed compound structure, and the integral ratio of the main protons (8H aromatic, 4H oxirane, 8H central methylene) was consistent with the theoretical number of protons in the molecule.

Proton NMR spectra confirm the successful synthesis of intermediates and final epoxy resin by identifying key chemical shifts corresponding to aldehyde, phenol, and epoxy functionalities.

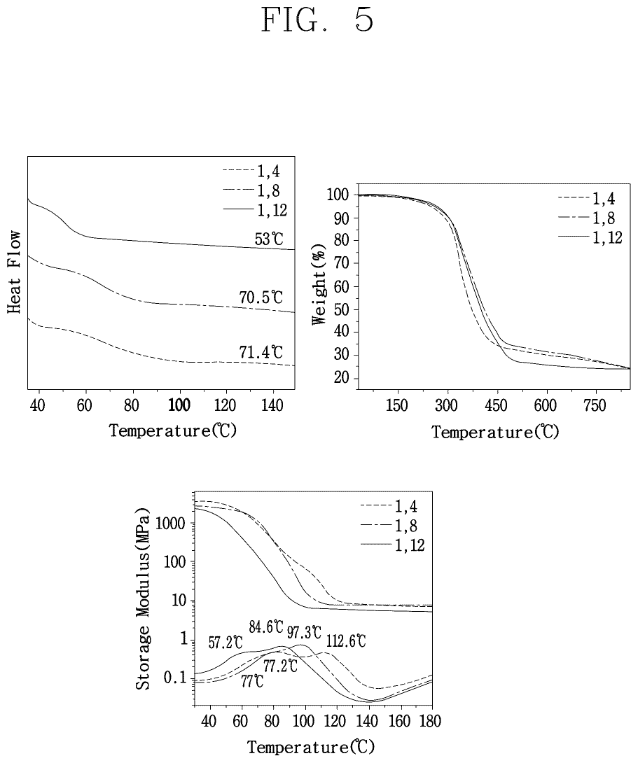

FIG. 5 illustrates DSC, TGA and DMA data of epoxy Films in accordance with an embodiment of the present invention. Differential Scanning calorimetry (DSC) analysis confirms the tunability of the glass transition temperature (Tg) in the epoxy vitrimer system, with values of 71.4° C. for C-4, 70.5° C. for C-8, and 53° C. for C-12, demonstrating the influence of chain length on thermal properties. Thermogravimetric Analysis (TGA) further highlights the material's high thermal stability, with degradation onset temperatures exceeding 250° C., ensuring robustness under high-temperature conditions. Additionally, Dynamic Mechanical Analysis (DMA) reveals excellent mechanical performance at elevated temperatures, with storage modulus peaks of 112.6° C. (C-4), 97.3° C. (C-8), and 84.6° C. (C-12), indicating strong thermal resistance and structural integrity across different formulations.

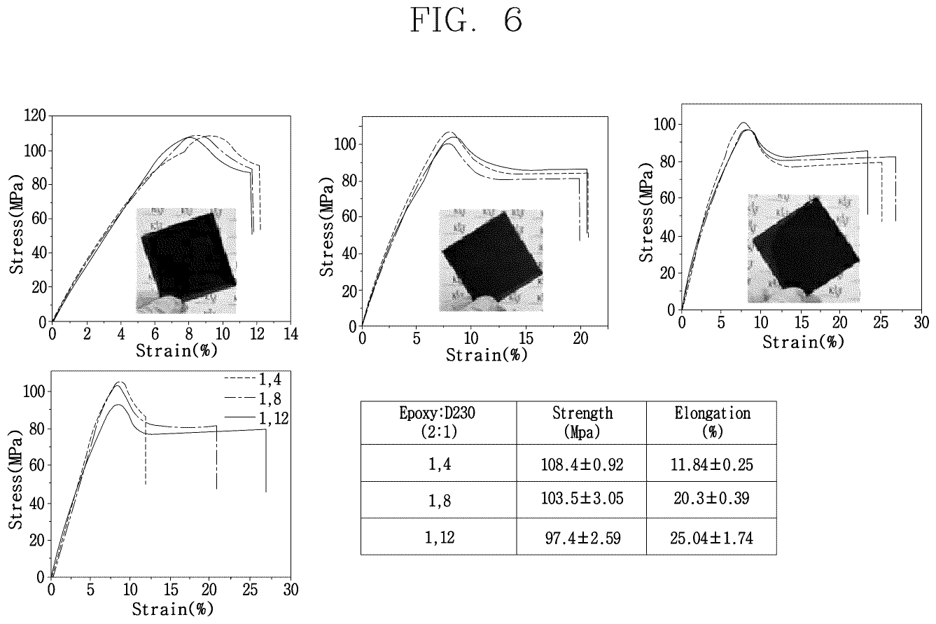

FIG. 6 illustrates Stress-strain curves of different epoxy with jeffamine D230 in accordance with an embodiment of the present invention. The mechanical properties of the synthesized epoxy vitrimers demonstrate a strong correlation between the carbon linker chain length and both tensile strength and elongation. The C-4 epoxy vitrimer exhibits the highest tensile strength of 108.4 MPa with a lower elongation of 11.84%, indicating a more rigid and highly crosslinked network. As the chain length increases, the C-8 vitrimer shows a slightly reduced tensile strength of 103.5 MPa but improved elongation of 20.3%, suggesting enhanced flexibility due to increased segmental mobility. The C-12 vitrimer further emphasizes this trend, displaying the lowest tensile strength of 97.4 MPa but the highest elongation of 25.04%, signifying a more flexible and ductile material. These variations highlight the tunability of the epoxy vitrimer system, allowing for precise control over mechanical properties to suit specific application requirements.

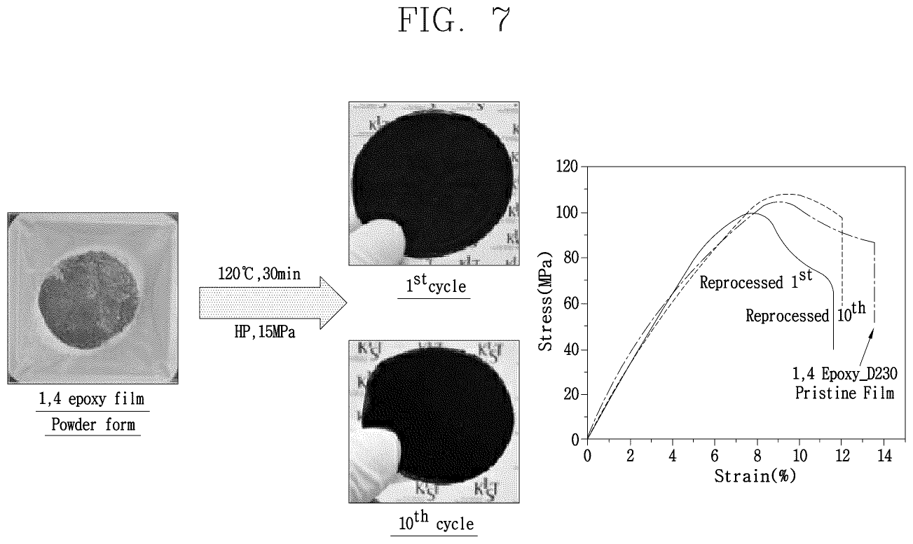

FIG. 7 illustrates 1,4 epoxy D230 Film Reprocessing up to 10 cycles in accordance with an embodiment of the present invention. The reprocessing was carried out by grinding the epoxy film using a mechanical mixture grinder. The resulting powdered sample was then reprocessed by hot pressing at 120° C. for 30 minutes under a pressure of 15 MPa. After 10 cycles, the image of the reprocessed film is shown in FIG. 7. The reprocessable epoxy vitrimer exhibits outstanding durability, retaining its mechanical integrity even after 10 thermal reprocessing cycles, with only a minimal loss in tensile strength and elasticity.

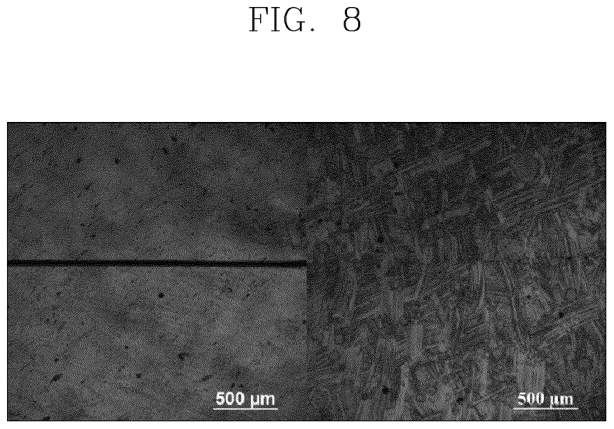

FIG. 8 illustrates self-healing of 1,4 epoxy D230 Film in accordance with an embodiment of the present invention. For self-healing, the film was stretched using a stretcher machine with a 7N force, and the image was captured using an optical microscope. Following this, the scratched film was placed in an oven at 120° C. for 30 minutes. The film was fully healed after this treatment, as shown in FIG. 8.

The epoxy vitrimer according to the present invention may realize self-healing functionality based on a dynamic covalent bond network formed through dual imine functionalization. The imine bond (—C═N—) has a reversible (dynamic covalent) property and may induce bond exchange reactions under external stimuli (such as heat and mild pressure).

During the self-healing process, the imine bonds present in the polymer matrix may undergo reversible dissociation of bonds around the crack interface under relatively low temperature conditions, such as 120° C., and subsequently reform new bonds between freely mobile molecular chains, thereby spontaneously restoring the damaged network. Such bond exchange reactions may proceed without an external catalyst, which may provide significant advantages in terms of simplification of the manufacturing process and cost reduction.

In addition, the kinetic characteristics of the imine bond may temporarily increase the mobility of the network during self-healing, enabling rapid and effective filling of microcracks or surface damage regions. In practice, the epoxy film of the present invention was experimentally confirmed to achieve complete structural recovery after heat treatment at 120° C. for 30 minutes with respect to artificial cracks generated under a load of 7 N.

Consequently, the self-healing mechanism exhibited in the embodiments of the present invention relies on the principle of reversible network reconstruction based on dynamic imine bonds, thereby ensuring not only the retention of key matter properties such as mechanical strength and elongation under repeated damage, but also long-term durability and environmental sustainability.

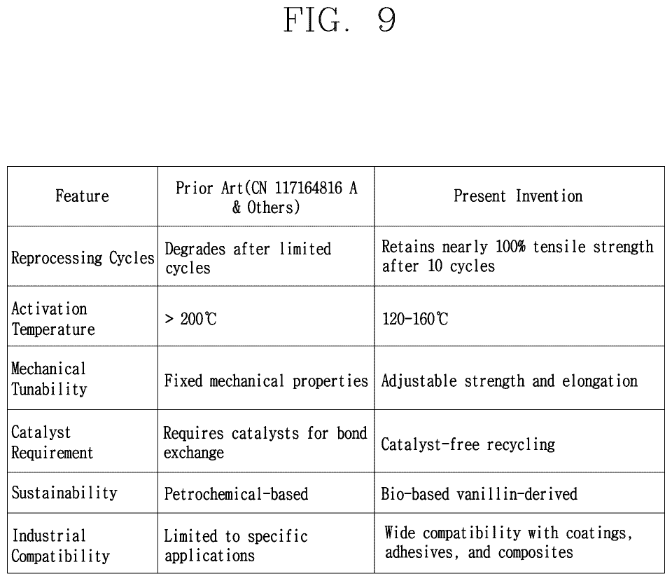

FIG. 9 illustrates a table which describe differences of the present invention with the Prior Art (CN 117164816 A& Others) in accordance with an embodiment of the present invention. Several existing epoxy vitrimer systems, including those disclosed in patents such as CN 117164816 A, primarily rely on Schiff-base chemistries or transesterification-based networks. These prior systems exhibit significant weaknesses that limit their practical applicability in industrial settings. There are following highlight key weaknesses in prior art and the present invention overcomes these limitations:

Limited Recyclability and Reprocessing Efficiency: Prior systems, such as CN 117164816 A, suffer from inefficient recyclability due to the rigidity of their Schiff-base epoxy networks. While these systems demonstrate some degree of reversibility, repeated reprocessing results in mechanical property degradation, making them unsuitable for extended use. The dual imine functionalization in the epoxy resins allows for high-efficiency reprocessing, retaining nearly 100% tensile strength and 85% elasticity even after ten thermal reprocessing cycles. This ensures long-term material usability and reduced waste generation.

High Activation Temperature for Bond Exchange Reactions: Many prior epoxy vitrimer systems require high activation temperatures (>200° C.) to enable reprocessing, limiting their applications in industries where low-temperature processing is critical. The dynamic imine bonds in our resin enable bond exchange reactions at significantly lower temperatures (120-160° C.), facilitating energy-efficient recycling and reprocessing.

Limited Mechanical Tunability and Strength Retention: Many prior vitrimer systems fail to offer tunable mechanical properties, resulting in brittle materials or materials with suboptimal strength. By varying the carbon linker chain length (C-3, C-4, C-8, C-12), our formulation allows precise control over tensile strength (97-108 MPa) and elongation (11-25%), making it adaptable for diverse industrial applications. Additionally, our epoxy film exhibits self-healing properties, making it beneficial for use in the fields of protective coatings, aerospace, automotive, electronics, and biomedical applications.

Catalyst Dependency in Conventional Vitrimers: Prior epoxy vitrimers often require external catalysts for dynamic covalent bond exchange, increasing processing complexity and cost, whereas the epoxy resin eliminates the need for catalysts by leveraging imine-based dynamic covalent bonds, simplifying manufacturing and reducing material costs.

Environmental and Sustainability Concerns: Many previously reported epoxy thermosets are derived from petroleum-based feedstocks, leading to significant environmental impact and poor biodegradability, whereas the system utilizes bio-based vanillin-derived aldehydes, enhancing sustainability while maintaining high-performance characteristics. This makes our resin an environmentally friendly alternative to conventional petrochemical-derived epoxy resins.

Compatibility with Industrial Processing Techniques: Prior art systems often struggle with compatibility for injection molding, 3D printing, and composite manufacturing due to their rigid crosslinked structures, whereas the epoxy resins integrate seamlessly with conventional industrial processing techniques, enabling large-scale production for coatings, adhesives, composites, and aerospace applications.

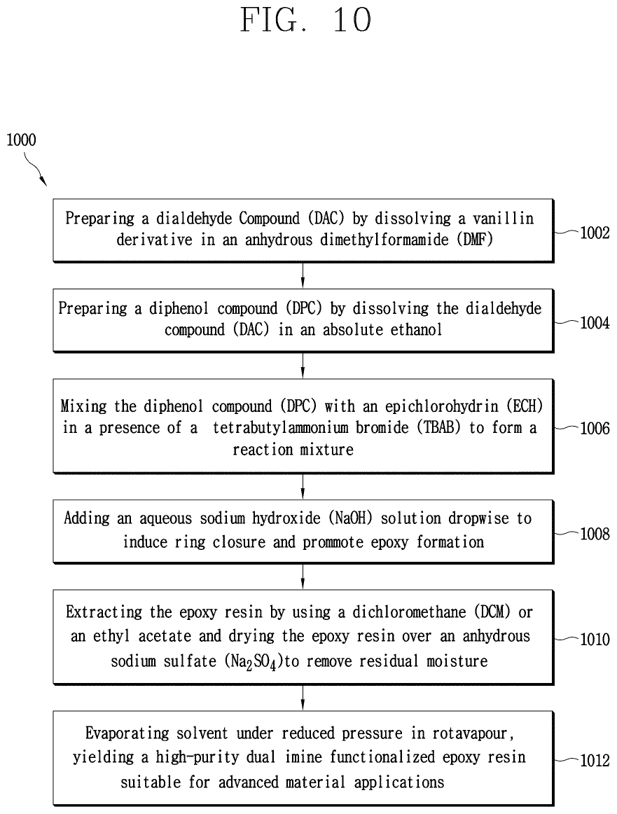

FIG. 10 illustrates a flow chart for process for synthesis of epoxy resin from vanillin-based dialdehydes, aminophenol derivatives, and epichlorohydrin in accordance with an embodiment of the present disclosure. The process 1000 includes:
Step 1002 discloses about preparing a dialdehyde compound (DAC) by dissolving a vanillin derivative in an anhydrous dimethylformamide (DMF);
Step 1004 discloses about preparing a diphenol compound (DPC) by dissolving the dialdehyde compound (DAC) in an absolute ethanol;
Step 1006 discloses about mixing the diphenol compound (DPC) with an epichlorohydrin (ECH) in a presence of a tetrabutylammonium bromide (TBAB) to form a reaction mixture; Step 1008 discloses about adding an aqueous sodium hydroxide (NaOH) solution dropwise to induce ring closure and promote epoxy formation;
Step 1010 discloses about extracting the epoxy resin by using a dichloromethane (DCM) or an ethyl acetate and drying the epoxy resin over an anhydrous sodium sulfate (Na₂SO₄) to remove residual moisture;
Step 1012 discloses about evaporating solvent under reduced pressure in a rotavapour, yielding a high-purity dual imine functionalized epoxy resin suitable for advanced material applications.

In another embodiment, the preparing the dialdehyde compound (DAC) by using the vanillin derivative in the anhydrous dimethylformamide (DMF), said preparing process comprising: dissolving the vanillin derivative in the anhydrous dimethylformamide (DMF) to form a mixture and stirring the mixture to ensure complete solubility; adding an alkyl dibromides and potassium carbonate (K₂CO₃) to the mixture; heating the mixture at 70° C. for 24 hours under an inert nitrogen atmosphere to prevent oxidation and unwanted side reactions.

In another embodiment, the preparing the dialdehyde compound (DAC) by using the vanillin derivative in the anhydrous dimethylformamide (DMF), said preparing process further comprising cooling the mixture to room temperature and pouring the mixture into a deionized water to precipitate the dialdehyde compound; vacuum filtering the precipitate of the dialdehyde compound and washing thoroughly to remove residual salts and unreacted reagents; purifying the precipitate of the dialdehyde compound via recrystallization in an ethanol or methanol to obtain a high-purity dialdehyde compound.

In another embodiment, the potassium carbonate (K₂CO₃) acts as a base in the mixture to facilitate a nucleophilic substitution reaction.

In another embodiment, the preparing the diphenol compound (DPC) by dissolving the dialdehyde compound (DAC) in the absolute ethanol, said preparing process comprising dissolving the dialdehyde compound (DAC) in the absolute ethanol to form a mixture and stirring the mixture to ensure complete dissolution; adding a p-Aminophenol gradually under continuous stirring into the mixture; heating the mixture to 80° C. under reflux for 24 hours to form an imine (—C═N—) linkages while ensuring complete conversion of starting materials.

In another embodiment, the preparing the diphenol compound (DPC) by dissolving the dialdehyde compound (DAC) in the absolute ethanol, said preparing process further comprising: cooling the mixture to room temperature and subjecting the mixture to vacuum filtration to separate a solid diphenol compound; washing the solid diphenol compound thoroughly with a cold ethanol to remove any unreacted residues and by-products; and vacuum drying the diphenol intermediate at 50-60° C. to obtain a high-purity diphenol compound (DPC).

In another embodiment, a condensation reaction is facilitated between aldehyde and amine functional groups.

In another embodiment, the tetrabutylammonium bromide (TBAB) acts as a phase-transfer catalyst to enhance an etherification reaction.

In another embodiment, the reaction mixture is maintained at 100-110° C. under continuous stirring to ensure complete functionalization.

In another embodiment, temperature of the reaction mixture is gradually increased to 50-60° C. to facilitate polymerization.

In addition, the epoxy resin synthesized by the synthesis method described above may have the following chemical formula:

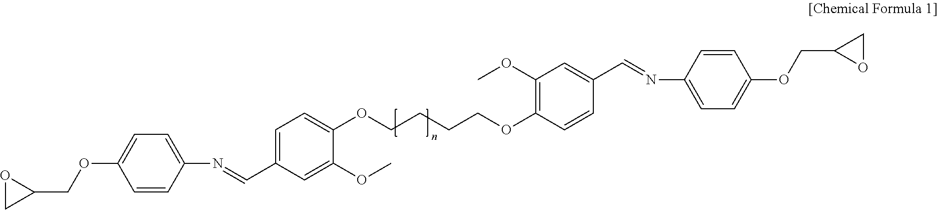

Chemical Formula 1 above may have the chemical name of 1,n-bis{2-[(4-(2,3-epoxypropoxy)benzylidene)amino]-5-methoxyphenoxy}alkane.

Specifically, the epoxy resin may correspond to any one selected from 1,2-bis{2-[(4-(2,3-epoxypropoxy)benzylidene)amino]-5-methoxyphenoxy}ethane, 1,3-bis{2-[(4-(2,3-epoxypropoxy)benzylidene)amino]-5-methoxyphenoxy}propane, and 1,4-bis{2-[(4-(2,3-epoxypropoxy)benzylidene)amino]-5-methoxyphenoxy}butane.

The present invention provides a bifunctional epoxy vitrimer synthesized from vanillin-derived bio-based materials, offering a sustainable alternative to conventional single-use epoxy thermosets. Unlike traditional epoxy resins, the developed vitrimer can be reprocessed multiple times without compromising mechanical integrity, retaining its tensile strength even after ten reprocessing cycles. The vanillin-based bifunctional epoxy (VBFE) was synthesized through a three-step reaction involving vanillin-derived dialdehyde, diol, and epoxy formation. To tailor mechanical properties, four epoxy vitrimers with varying carbon linker chain lengths (C-4, C-8, C-12) were synthesized, enabling precise control over strength and stretchability. Dynamic covalent imine bonds impart excellent reprocessability and self-healing properties, allowing autonomous crack repair within 30 minutes at 120° C. under mild pressure. Differential scanning calorimetry (DSC) and dynamic mechanical analysis (DMA) confirmed tunable glass transition temperatures (50° C.-75° C.) and high thermal stability. Furthermore, the vitrimer exhibits nearly 100% self-healability, ensuring extended material lifespan and reduced waste. The integration of bio-derived feedstocks and dynamic covalent networks enhances both sustainability and performance, making this vitrimer a promising candidate for advanced applications in coatings, adhesives, electronic encapsulants, and high-performance structural composites.

The present invention introduces a dual imine functionalized epoxy resin system with superior recyclability, self-healing, and enhanced mechanical performance. The use of vanillin-derived aldehyde precursors not only improves sustainability but also offers tunable mechanical and thermal properties suitable for various industrial applications. Unlike conventional epoxy resins, which suffer from irreversibility and poor reprocessing, the present system provides a cost-effective and environmentally friendly alternative. The integration of dynamic imine bonds allows for multiple reprocessing cycles without significant property loss, addressing key challenges in thermoset recycling. Additionally, the self-healing capability ensures prolonged material lifespan, reducing waste and maintenance costs. These advantages make this resin system highly applicable in high-performance coatings, adhesives, composite materials, and aerospace or automotive components. By leveraging renewable feedstocks and innovative chemical strategies, this invention sets a new standard for sustainable and high-performance epoxy materials

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible.

We claim:

1. A process for synthesis of epoxy resin from vanillin-based dialdehydes, aminophenol derivatives and epichlorohydrin, said process comprising:
preparing a dialdehyde compound (DAC) by dissolving a vanillin derivative in an anhydrous dimethylformamide (DMF);
preparing a diphenol compound (DPC) by dissolving the dialdehyde compound (DAC) in an absolute ethanol;
mixing the diphenol compound (DPC) with an epichlorohydrin (ECH) in a presence of a tetrabutylammonium bromide (TBAB) to form a reaction mixture;
adding an aqueous sodium hydroxide (NaOH) solution dropwise to induce ring closure and promote epoxy formation;
extracting the epoxy resin by using a dichloromethane (DCM) or an ethyl acetate and drying the epoxy resin over an anhydrous sodium sulfate (Na₂SO₄) to remove residual moisture;
evaporating solvent under reduced pressure in a rotavapour, yielding a high-purity dual imine functionalized epoxy resin suitable for advanced material applications.

2. The process as claimed in claim 1, wherein the preparing the dialdehyde compound (DAC) by using the vanillin derivative in the anhydrous dimethylformamide (DMF), said preparing process comprising:
dissolving the vanillin derivative in the anhydrous dimethylformamide (DMF) to form a mixture and stirring the mixture to ensure complete solubility;
adding an alkyl dibromides and potassium carbonate (K₂CO₃) to the mixture;
heating the mixture at 70° C. for 24 hours under an inert nitrogen atmosphere to prevent oxidation and unwanted side reactions.

3. The process as claimed in claim 2, wherein the preparing the dialdehyde compound (DAC) by using the vanillin derivative in the anhydrous dimethylformamide (DMF), said preparing process further comprising:
cooling the mixture to room temperature and pouring the mixture into a deionized water to precipitate the dialdehyde compound;
vacuum filtering the precipitate of the dialdehyde compound and washing thoroughly to remove residual salts and unreacted reagents;
purifying the precipitate of the dialdehyde compound via recrystallization in an ethanol or methanol to obtain a high-purity dialdehyde compound.

4. The process as claimed in claim 2, wherein the potassium carbonate (K₂CO₃) acts as a base in the mixture to facilitate a nucleophilic substitution reaction.

5. The process as claimed in claim 1, wherein the preparing the diphenol compound (DPC) by dissolving the dialdehyde compound (DAC) in the absolute ethanol, said preparing process comprising:
dissolving the dialdehyde compound (DAC) in the absolute ethanol to form a mixture and stirring the mixture to ensure complete dissolution;
adding a p-Aminophenol gradually under continuous stirring into the mixture;
heating the mixture to 80° C. under reflux for 24 hours to form an imine (—C═N—) linkages while ensuring complete conversion of starting materials.

6. The process as claimed in claim 5, wherein the preparing the diphenol compound (DPC) by dissolving the dialdehyde compound (DAC) in the absolute ethanol, said preparing process further comprising:
cooling the mixture to room temperature and subjecting the mixture to vacuum filtration to separate a solid diphenol compound;
washing the solid diphenol compound thoroughly with a cold ethanol to remove any unreacted residues and by-products; and
vacuum drying the diphenol intermediate at 50-60° C. to obtain a high-purity diphenol compound (DPC).

7. The process as claimed in claim 5, wherein a condensation reaction is facilitated between aldehyde and amine functional groups.

8. The process as claimed in claim 1, wherein the tetrabutylammonium bromide (TBAB) acts as a phase-transfer catalyst to enhance an etherification reaction.

9. The process as claimed in claim 1, wherein the reaction mixture is maintained at 100-110° C. under continuous stirring to ensure complete functionalization.

10. The process as claimed in claim 1, wherein temperature of the reaction mixture is gradually increased to 50-60° C. to facilitate polymerization.

11. An epoxy resin prepared according to claim 1, the epoxy resin having the following chemical formula,

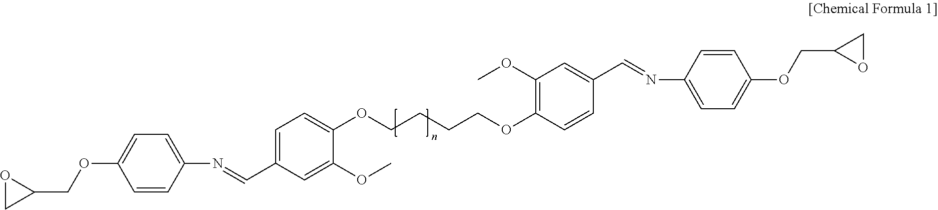

12. The epoxy resin as claimed in claim 11, wherein the epoxy resin is any one selected from 1,2-bis{2-[(4-(2,3-epoxypropoxy)benzylidene)amino]-5-methoxyphenoxy}ethane, 1,3-bis{2-[(4-(2,3-epoxypropoxy)benzylidene)amino]-5-methoxyphenoxy}propane, and 1,4-bis{2-[(4-(2,3-epoxypropoxy)benzylidene)amino]-5-methoxyphenoxy}butane.
